(19) United States
(12) Patent Application Publication
(10) Pub. No.: US 2026/0061008 A1
(43) Pub. Date: Mar. 5, 2026

(54) METHODS OF INCREASING MESENCHYMAL STROMAL CELL BIOSYNTHESIS OF SPECIALIZED PRO-RESOLVING MEDIATORS AND METHODS OF USE

(71) Applicants: University of Oregon, Eugene, OR (US); Georgia Tech Research Corporation, Atlanta, GA (US)

(72) Inventors: Nick Willett, Eugene, OR (US); Elaine deLorimier, Springfield, OR (US); Edward Botchwey, Atlanta, GA (US); Thomas Turner, Atlanta, GA (US)

(73) Assignees: University of Oregon, Eugene, OR (US); Georgia Tech Research Corporation, Atlanta, GA (US)

(21) Appl. No.: 19/105,614

(22) PCT Filed: Aug. 24, 2023
(86) PCT No.: PCT/US2023/072790

§ 371 Date: Feb. 21, 2025

Related U.S. Application Data
(60) Provisional application No. 63/400,512, filed on Aug. 24, 2022.

(51) Int. Cl. A61K 35/28; A61K 31/202; A61P 19/02; C12P 7/6427

(52) U.S. Cl. CPC A61K 35/28; A61K 31/202; A61P 19/02; C12P 7/6427

(57) ABSTRACT

Methods of stimulating production of specialized pro-resolving mediators (SPMs) by mesenchymal stem cells (MSCs) are provided. The methods include contacting MSCs with one or more SPM fatty acid precursors, SPM intermediates, or a combination of two or more thereof. Also provided are compositions including the stimulated MSCs or conditioned media from the stimulated MSCs. Methods of treating or inhibiting inflammation are also provided, including administering to a subject stimulated MSCs or conditioned media from stimulated MSCs.

### CROSS REFERENCE TO RELATED APPLICATIONS

This application claims priority to U.S. Provisional Application No. 63/400,512, filed Aug. 24, 2022, which is incorporated by reference in its entirety.

### SEQUENCE LISTING INCORPORATION BY REFERENCE

The Sequence Listing is submitted as an XML file in the form of the file named 1505-108906-02_Sequence_Listing.xml (2,845 bytes), which was created on Aug. 22, 2023, which is incorporated by reference herein.

### FIELD

The present disclosure relates to methods of increasing production of specialized pro-resolving mediators (SPMs) by mesenchymal stromal cells (MSCs) and methods and compositions for treating inflammation and/or promoting tissue regeneration using MSCs.

### BACKGROUND

Osteoarthritis (OA) is the most common joint disorder in the United States, affecting roughly 12% of adults over the age of 25 and costing the US economy over $60 billion per year. Individuals with OA have altered joint function, articular cartilage degradation and proteoglycan loss, chondrocyte hypotrophy, osteophyte formation, and bone remodeling, ultimately progressing to bone-on-bone contact due to extensive cartilage loss. OA leads to chronic pain, joint dysfunction, stiffness, loss of mobility and inflammation. Current methods to treat OA are limited to conventional weight loss and exercise approaches, managing symptomatic pain with non-steroidal anti-inflammatory drugs (NSAIDs), or intra-articular steroid injections. While NSAIDs reduce pain in OA, they do not treat the disease itself and provide no therapeutic benefit to prevent or reverse cartilage degradation, proteoglycan loss, or other hallmarks of OA. Steroid injections have been shown to worsen OA disease progression, potentially due to associated cell death. With symptomatic management, OA will continue to progress in severity and eventually require joint reconstruction or replacement surgeries. Therefore, there is a significant need for non-surgical regenerative therapies for OA that can regenerate joint tissues, reduce pain, and restore function.

### SUMMARY

Provided herein are methods of stimulating or increasing production of specialized pro-resolving mediators (SPMs) by MSCs. The disclosed methods and related compositions can be used in treatment of inflammation and/or promoting tissue regeneration.

Described herein are methods to enhance MSC-mediated production of a class of fatty-acid derived molecules that are known to reduce or resolve inflammation, called specialized pro-resolving mediators (SPMs), inclusive of resolvins, maresins, lipoxins, and protectins, among others. MSC and cell therapy production of such SPMs can enhance inflammation resolution, promote tissue regeneration, and reduce degeneration. Additional potential benefits include functional improvements and reduction of pain.

Provided herein are methods of stimulating or increasing production of one or more specialized pro-resolving mediators (SPMs) by mesenchymal stromal cells (MSCs), including contacting a population of MSCs with one or more SPM fatty acid precursors or SPM intermediates, or a combination of two or more thereof, thereby stimulating or increasing production of SPMs (for example, compared to a control). In some aspects, the one or more SPM fatty acid precursors is selected from the group consisting of docosahexaenoic acid (DHA), eicosapentaenoic acid (EPA), arachidonic acid (AA), and docosapentaenoic acid (DPA). In one example, the SPM fatty acid precursor is DHA. In other aspects, the one or more SPM intermediates is selected from the group consisting of 17(S)-hydroxy-docosahexaenoic acid, 17(S)-hydroxy-hydroperoxide docosahexaenoic acid (HpDHA), 4(S)-hydroperoxy-17(S) hydroxy-docosahexaenoic acid, 4(S)-5(S)-epoxy-17(S)-hydroxy-docosahexaenoic acid, 7(S),8(S)-epoxy-17(S)-hydroxy-docosahexaenoic acid, 15(S)-hydroperoxy-eicosapentaenoic acid, 5(S)-Hydroperoxy-15(S)-hydroxyeicosapentanoic acid, 18(R)-hydroperoxy-eicosapentaenoic acid, 5(S)-hydroperoxy-18(R)-hydroxyeicosapentanoic acid, 5(S),6(S)-epoxy-18(R)-hydroxyeicosapentanoic acid, 15(S)-Hydroperoxy eicosatetraenoic acid, 5(S), 15(S)-Di-Hydroperoxide eicosatetraenoic acid, 5(S),6(S)-epoxy-15(S) Di-Hydroperoxide eicosatetraenoic acid, and 13(R)-Hydroperoxy docosapentaenoic acid (HpDPA). In additional aspects, the SPMs are selected from one or more resolvins (for example, one or more of resolvin D1, resolvin D2, resolvin D3, resolvin D4, resolvin D5, resolvin D6, aspirin-triggered resolvin D1, resolvin E1, resolvin E2, resolvin E3, resolvin E4, resolvin T1, resolvin T2, resolvin T3, and resolvin T4), lipoxins (for example, one or more of lipoxin A₄, lipoxin A₅, lipoxin B₄, and 15-epi-lipoxin A₄), protectins (for example, one or more of protectin (PDX), PCTR1, PCTR2, and PCTR3), maresins (such as one or more of maresin 1, maresin 2, MCTR1, MCTR2, and MCTR3), or a combination of two or more thereof. In one example, the resolvin is resolvin D1.

In some aspects, the MSCs are contacted with the one or more SPM fatty acid precursors, SPM intermediates, or a combination of two or more thereof for about 3-48 hours. In additional aspects, the MSCs are human MSCs. In some aspects, the methods further include collecting conditioned media from the MSCs contacted with the one or more SPM fatty acid precursors, SPM intermediates, or a combination of two or more thereof.

Also provided are compositions including MSCs treated or produced by the methods described herein, for example compositions including the treated MSCs and a pharmaceutically acceptable carrier. In some aspects the carrier is a hydrogel, ceramic, a polymer, a sponge, or a scaffold. In some examples, the carrier is selected from alginate, polyethylene glycol, hyaluronic acid, and collagen. In some aspects, the carrier is a hydrogel including polyethylene glycol or functionalized polyethylene glycol (such as polyethylene glycol functionalized with one to four maleimide moieties).

Also provided are methods of treating or inhibiting inflammation in a subject. In some aspects, the methods include administering to the subject mesenchymal stromal cells (MSCs) treated with or produced by the methods described herein, or a composition including the treated MSCs. In other aspects, the methods include administering to the subject mesenchymal stromal cells (MSCs) and one or more specialized pro-resolving molecule (SPM) fatty acid precursors or SPM intermediates, or a combination of two or more thereof. In still further aspects, the methods include administering to the subject conditioned media from MSCs produced by the methods described herein, thereby treating or inhibiting the inflammation in the subject.

In some aspects, the one or more SPM fatty acid precursors is selected from the group consisting of docosahexaenoic acid (DHA), eicosapentaenoic acid (EPA), arachidonic acid (AA), and docosapentaenoic acid (DPA). In one example, the SPM fatty acid precursor is DHA. In other aspects, the one or more SPM intermediates is selected from the group consisting of 17(S)-hydroxy-docosahexaenoic acid, 17(S)-hydroxy-hydroperoxide docosahexaenoic acid (HpDHA), 4(S)-hydroperoxy-17(S) hydroxy-docosahexaenoic acid, 4(S)-5(S)-epoxy-17(S)-hydroxy-docosahexaenoic acid, 7(S),8(S)-epoxy-17(S)-hydroxy-docosahexaenoic acid, 15(S)-hydroperoxy-eicosapentaenoic acid, 5(S)-Hydroperoxy-15(S)-hydroxyeicosapentanoic acid, 18(R)-hydroperoxy-eicosapentaenoic acid, 5(S)-hydroperoxy-18(R)-hydroxyeicosapentanoic acid, 5(S),6(S)-epoxy-18(R)-hydroxyeicosapentanoic acid, 15(S)-Hydroperoxy eicosatetraenoic acid, 5(S), 15(S)-Di-Hydroperoxide eicosatetraenoic acid, 5(S),6(S)-epoxy-15(S) Di-Hydroperoxide eicosatetraenoic acid, and 13(R)-Hydroperoxy docosapentaenoic acid (HpDPA). In additional aspects, the SPMs are selected from one or more resolvins (for example, one or more of resolvin D1, resolvin D2, resolvin D3, resolvin D4, resolvin D5, resolvin D6, aspirin-triggered resolvin D1, resolvin E1, resolvin E2, resolvin E3, resolvin E4, resolvin T1, resolvin T2, resolvin T3, and resolvin T4), lipoxins (for example, one or more of lipoxin A₄, lipoxin Aₛ, lipoxin B₄, and 15-epi-lipoxin A₄), protectins (for example, one or more of protectin (PDX), PCTR1, PCTR2, and PCTR3), maresins (such as one or more of maresin 1, maresin 2, MCTR1, MCTR2, and MCTR3), or a combination of two or more thereof. In one example, the resolvin is resolvin D1.

In some examples, the MSCs, the one or more specialized pro-resolving molecule (SPM) fatty acid precursors or SPM intermediates, or a combination of two or more thereof, or both are formulated in a pharmaceutically acceptable carrier. In some aspects the carrier is a hydrogel, ceramic, a polymer, a sponge, or a scaffold. In some examples, the carrier is selected from alginate, polyethylene glycol, hyaluronic acid, and collagen. In some aspects, the carrier is a hydrogel including polyethylene glycol or functionalized polyethylene glycol (such as polyethylene glycol functionalized with one to four maleimide moieties).

In some aspects, the MSCs are human MSCs. In some examples, the MSCs are autologous to the subject. In some examples, the subject has inflammation resulting from a musculoskeletal disorder or injury, or a dermal injury. In some examples, the musculoskeletal disorder or injury includes osteoarthritis, degenerative disc disease or disc injury, joint injury, bone fracture, muscle injury or tear, ligament injury or tear, or tendon injury or tear. In other examples, the subject has an autoimmune disease or disorder selected from rheumatoid arthritis, graft-versus-host-disease, Crohn's disease, arthritis, inflammatory bowel disease, or scleroderma. In some aspects, the MSCs or conditioned media is administered to the subject at or near the site of inflammation or injury. In one example, the MSCs or conditioned media is administered to the subject intra-articularly.

The foregoing and other features of the disclosure will become more apparent from the following detailed description, which proceeds with reference to the accompanying figures.

### BRIEF DESCRIPTION OF THE DRAWINGS

FIGS. 1A-1D illustrate increased resolvin D1 (RvD1) production by MSCs treated with docosahexaenoic acid (DHA). FIG. 1A is a schematic diagram illustrating the experimental method. FIGS. 1B and 1C are graphs showing RvD1 production by MSCs at the indicated time after treatment with the indicated amount of DHA. a and b are statistically different from 0 μM DHA groups. FIG. 1D is a graph showing RvD1 production by MSCs from four different donors after treatment with 80 μM DHA for 24 hours. * RvD1 levels from each donor are significantly different from untreated MSCs.

FIGS. 2A-2C show cytokine secretome changes in hMSCs after DHA treatment. FIG. 2A is a heatmap showing changes in cytokines in hMSCs treated with the indicated concentrations of DHA. FIG. 2B is a plot showing a Partial Least Squares Analysis (PLSDA) assessment of the overall effect of the DHA treatment on hMSC secretion of cytokines, showing distinct separation of the hMSC groups according to LV1 with the high DHA treatment to the left and no DHA treatment to the right. FIG. 2C is a graph showing a LUMINEX analysis of cytokine expression levels from DHA treated hMSCs compared to untreated hMSCs. Cytokine levels broadly increased in DHA treated hMSCs; these included numerous immunomodulatory cytokines and growth factors thought to be critical to MSC therapeutic efficacy including GM-CSF, GRO, and IL-4 among others.

FIGS. 3A-3D illustrate RvD1 levels from MSCs encapsulated in PEG-4MAL. FIG. 3A shows RvD1 levels from MSCs seeded in TCP and two conditions of DHA treatment in PEG-4MAL. Group A: DNA added to cell culture medium immediately after gel formation; Group B: DNA added to the cell culture medium 24 hours after gel formation. FIG. 3B shows RvD1 levels from TCP seeded MSCs over a period of four days. FIGS. 3C and 3D show RvD1 levels from MSCs encapsulated in PEG-4MAL with DHA treatment Groups A and B, respectively.

FIGS. 4A-4C show lipidomics measurement of SPMs secreted from DHA-treated MSCs. FIGS. 4A shows D-Series Resolvin levels in cell culture media from MSCs cultured in TCP and encapsulated in PEG. FIG. 4B shows Maresin 2 levels in cell culture media from MSCs cultured in TCP and encapsulated in PEG. FIG. 4C shows 17-HDHA intermediate levels in culture media from MSCs cultured in TCP and encapsulated in PEG.

FIGS. 5A and 5B illustrate an exemplary method for evaluating RvD1-secreting MSCs in a rat model of osteoarthritis (OA). FIG. 5A is a schematic diagram illustrating a timeline for the experiment. Rats are treated with saline (control), untreated MSCs, or DHA-treated MSCs 24 hours after medial meniscal transection (MMT) surgery. FIG. 5B shows the MMT model, which leads to OA symptoms in rats.

### SEQUENCE LISTING

The nucleic and amino acid sequences listed in the accompanying sequence listing are shown using standard letter abbreviations for nucleotide bases, and single letter code for amino acids, as defined in 37 C.F.R. 1.822. For nucleic acids, only one strand of each nucleic acid sequence is shown, but the complementary strand is understood as included by any reference to the displayed strand.
SEQ ID NO: 1 is an exemplary “RGD” peptide: GCGYGRGDSPG
SEQ ID NO: 2 is an exemplary “VPM” peptide: GCRDVPMSMRGGDRCG

### DETAILED DESCRIPTION

OA is now recognized not simply as a disease of wear and tear on the joint but one of chronic unresolved inflammation, significantly contributing to disease progression and symptoms. Specialized pro-resolving mediators (SPMs) are a class of small lipids that act to reduce or resolve inflammation and/or promote tissue regeneration. Indeed, repeated intra-articular injections of Resolvin D1 (RvD1) into knee joints can attenuate OA progression in mice (Sun et al., Sci. Rep. 9:426, 2019), suggesting specialized pro-resolving lipid mediators are thus an attractive potential drug target for OA. However, these lipid mediators are innately unstable and are very challenging to manufacture in large quantities, limiting their pharmaceutical use. As demonstrated herein, a cell-based system utilizing Mesenchymal Stromal Cells (MSCs) treated in vitro with the precursor molecule for RvD1 can biosynthesize this SPM.

### I. Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “a molecule” includes singular or plural molecules and can be considered equivalent to the phrase “at least one molecule.” As used herein, the term “comprises” means “includes.” It is further to be understood that any and all base sizes or amino acid sizes, and all molecular weight or molecular mass values, are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Administering: To provide or give a subject an agent by any effective route. Exemplary routes of administration include, but are not limited to, oral, injection (such as intra-articular, subcutaneous, intramuscular, intradermal, intraperitoneal, and intravenous), sublingual, transdermal (either directly over the joint or systemically), topical, intranasal, and inhalation routes.

Biocompatible: Any material, that, when implanted in a mammalian subject, does not provoke an adverse response in the subject. A biocompatible material, when introduced into an individual, is able to perform its intended function, and is not toxic or injurious to that individual, nor does it induce immunological rejection of the material in the subject.

Control: A standard for comparison to a sample, for example, for the determination of differential activity. In one example, a control is a sample (such as MSCs) that is not treated with a compound (such as one or more SPM fatty acid precursors or SPM intermediates). In other examples, a control is a reference value based on a known or determined population value, and can be supplied in the format of a graph or table that permits comparison of measured, experimentally determined values.

Hydrogel: A network of polymer chains that are hydrophilic, sometimes found as a colloidal gel in which water is the dispersion medium. Hydrogels are highly absorbent natural or synthetic polymeric networks. Hydrogels also possess a degree of flexibility similar to natural tissue.

Mesenchymal stromal cells (MSCs): Spindle shaped plastic-adherent cells isolated from bone marrow, adipose, umbilical cord, dental pulp, or other sources, having multipotent differentiation capacity in vitro. Criteria defining MSCs are not fully settled. In some examples, MSCs have the following characteristics: 1) plastic-adherent when maintained in standard culture conditions in tissue culture flasks; 2) >95% of the population expresses CD105, CD73, and CD90 as measured by flow cytometry and ≤2% of the population expresses CD45, CD34, CD14 or CD11b, CD79a or CD19, and HLA class II; 3) the cells are capable of trilineage differentiation in vitro to osteoblasts, adipocytes, and chondrocytes; and 4) have immunomodulatory activity, e.g., T-cell suppression via induction of indoleamine 2,3-dioxygenase activity by IFN-γ±TNF-α (Dominici et al., Cytotherapy 8:315-317, 2006; Krampera et al., Cytotherapy 15:1054-1061, 2013).

Osteoarthritis (OA): Osteoarthritis (OA) is a degenerative joint disease characterized by a fragmentation and erosion of the articular cartilage, which becomes soft, frayed and thinned with alteration of the subchondral bone, hypertrophy of the bone, including outgrowths of marginal osteophytes and changes accompanied by pain and stiffness, and finally by loss of function. Osteoarthritis mainly affects the weight bearing joints. When clinically evident, osteoarthritis is a major cause of morbidity and disability, especially for the elderly, due to joint pain, morning stiffness, and limitation of movement and commonly involves the neck, lower back, knees, hips and joints of the fingers. Osteoarthritis can also develop in joints that have suffered injury or trauma in the past, or have been subjected to prolonged heavy use.

Clinical indications of OA include, but are not limited to: pain (commonly in the hands, hips, knees, or feet, and sometimes in the spine), tenderness and occasional swelling of the joint, stiffness (for example, that lasting less than 1 hour) after long periods of inactivity (such as in the morning after a night's sleep or after sitting for a long time), limited motion of the joint, deformity of the joint (usually in later stages of osteoarthritis), cracking or “creaking” (crepitation) of the joints accompanied by pain, and other effects on the joint recognized by those of skill in the art.

Pharmaceutically acceptable carriers: Remington: The Science and Practice of Pharmacy, Adejare (Ed.), Academic Press, London, United Kingdom, 23^{rd} Edition (2021) describes carriers and formulations suitable for pharmaceutical delivery of the compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical preparations to be administered can contain minor amounts of non-toxic auxiliary substances, such as wetting or emulsifying agents, preservatives, and pH buffering agents and the like, for example sodium acetate or sorbitan monolaurate.

Specialized pro-resolving mediators (SPMs): A class of small lipids that act to reduce or resolve inflammation and/or promote tissue regeneration. SPMs are derived from polyunsaturated fatty acids, such as arachidonic acid (AA), eicosapentaenoic acid (EPA), docosahexaenoic acid (DHA), and docosapentaenoic acid (DPA). Classes of SPMs include lipoxins (derived from AA), E-series resolvins (derived from EPA), D-series resolvins, protectins, and maresins (derived from DHA), and T-series resolvins (derived from DPA). In some examples, SPMs are produced by MSCs.

Subject: Human and non-human animals, including vertebrates, such as mammals, such as non-human primates, mice, rats, rabbits, sheep, dogs, cats, horses, and cows. In some aspects of the described methods, the subject is a human.

Treating or inhibiting a disease: “Treating” refers to a therapeutic intervention that ameliorates a sign or symptom of a disease or pathological condition, such a sign or symptom of OA. Treatment can also induce remission or cure of a condition, such as OA. In particular examples, “inhibiting” a disease includes inhibiting the full development of a disease, such as delaying the development or severity of OA.

### II. Methods of Increasing Production of SPMs by SPCs

Provided are methods of stimulating or increasing production of one or more SPMs by MSCs, including contacting a population of MSCs with one or more SPM fatty acid precursors, one or more SPM intermediates, or a combination of two or more thereof, thereby stimulating or increasing production of SPMs. In some examples, the MSCs are contacted with one or more of the SPM fatty acid precursors or intermediates provided in Table 1.

In some examples, the one or more SPM fatty acid precursors is selected from docosahexaenoic acid (DHA), eicosapentaenoic acid (EPA), arachidonic acid (AA), and docosapentaenoic acid (DPA). In one example, the SPM fatty acid precursor is DHA. In other examples, the one or more SPM intermediates is selected from 17(S)-hydroxy-docosahexaenoic acid, 17(S)-hydroxy-hydroperoxide docosahexaenoic acid (HpDHA), 4(S)-hydroperoxy-17(S) hydroxy-docosahexaenoic acid, 4(S)-5(S)-epoxy-17(S)-hydroxy-docosahexaenoic acid, 7(S),8(S)-epoxy-17(S)-hydroxy-docosahexaenoic acid, 15(S)-hydroperoxy-eicosapentaenoic acid, 5(S)-Hydroperoxy-15(S)-hydroxyeicosapentanoic acid, 18(R)-hydroperoxy-eicosapentaenoic acid, 5(S)-hydroperoxy-18(R)-hydroxyeicosapentanoic acid, 5(S),6(S)-epoxy-18(R)-hydroxyeicosapentanoic acid, 15(S)-Hydroperoxy eicosatetraenoic acid, 5(S), 15(S)-Di-Hydroperoxide eicosatetraenoic acid, 5(S),6(S)-epoxy-15(S) Di-Hydroperoxide eicosatetraenoic acid, and 13(R)-Hydroperoxy docosapentaenoic acid (HpDPA).

In some examples, the MSCs are contacted with the one or more SPM fatty acid precursors or SPM intermediates, or a combination of two or more thereof for about 3-48 hours (for example, about 3 hours, about 6 hours, about 12 hours, about 18 hours, about 24 hours, about 36 hours, or about 48 hours). In one example, the MSCs are contacted with the one or more SPM fatty acid precursors, SPM intermediates, or a combination of two or more thereof for about 24 hours.

In some examples, the methods stimulate or increase production by MSCs of SPMs selected from one or more resolvins, lipoxins, protectins, maresins, or a combination of two or more thereof, compared to a control (for example, compared to untreated MSCs). In some examples, the methods result in a statistically significant increase in production by MSCs of one or more SPMs compared to the control (such as untreated MSCs).

In some examples, the one or more SPMs are selected from those listed in Table 1. In some examples, the SPM is a resolvin, for example, one or more of resolvin D1, resolvin D2, resolvin D3, resolvin D4, resolvin D5, resolvin D6, aspirin-triggered resolvin D1, resolvin E1, resolvin E2, resolvin E3, resolvin E4, resolvin T1, resolvin T2, resolvin T3, and resolvin T4. In a particular example, the resolvin is resolvin D1. In other examples, the SPM is a lipoxin, for example, one or more of lipoxin A₄, lipoxin A₅, lipoxin B₄, and 15-epi-lipoxin A₄. In further examples, the SPM is a protectin, for example, one or more of protectin (PDX), PCTR1, PCTR2, and PCTR3. In still further examples, the SPM is a maresin, for example, one or more of maresin 1, maresin 2, MCTR1, MCTR2, and MCTR3.

In particular examples, the MSCs are human MSCs. MSCs may be obtained directly from a subject or a donor from lipoaspirate, bone marrow aspirate, or another tissue source. Collected tissue then can be manipulated through micronizing, fragmenting, cutting, centrifuging, or other minimally manipulative procedures to isolate MSCs. Alternatively, the MSCs may be isolated by procedures such as enzymatic digestion (e.g., with collagenase), tissue culture plate adherence, cell sorting, or other, and expanded in culture. Cells may be isolated directly from a subject for autologous implantation or may be isolated from HLA-matched or unrelated mismatched donors.

In one specific example, the MSCs are contacted with DHA in order to stimulate production of one or more resolvins (such as resolvin D1). In some examples, the MSCs are contacted with about 50 μM to about 300 μM DHA, such as about 50 μM, about 60 μM, about 80 μM, about 100 μM, about 120 u M, about 140 μM, about 160 μM, about 180 μM, about 200 μM, about 240 μM, about 260 μM, about 280 μM, or about 300 μM DHA. In particular examples, the MSCs are contacted with about 80 μM DHA, about 160 μM DHA, or about 240 μM DHA. Appropriate amounts of SPM precursors or intermediates (such as those listed in Table 1) to stimulate SPM production by MSCs can be determined by one of skill in the art, for example, using the methods described in Example 1.

TABLE 1
Exemplary SPM precursors, intermediates, and enzymes

| Precursors: | Intermediates: | Enzymes: | SPMs: |
|---|---|---|---|
| Arachidonic | 15(S)-Hydroperoxy | Lipoxygenase-5 | LipoxinA₄ |
| Acid | eicosatetraenoic acid | (LOX5) | (LXA4) |
| | 5(S),15(S)-Di- | Lipoxygenase-15 | LipoxinA₅ |
| | Hydroperoxide | (LOX15) | LipoxinB₄ |
| | eicosatetraenoic acid | | 15-epi-LXA4 |
| | 5(S),6(S)-epoxy-15(S) | | |
| | Di- | | |
| | Hydroperoxide | | |
| | eicosatetraenoic acid | | |
| Docosapentaenoic | 13(R)-Hydroperoxy | Cycloxygenase-2 | ResolvinT1 |
| Acid | docosapentaenoic acid | (COX2) | ResolvinT2 |
| (DPA) | (HpDPA) | | ResolvinT3 |
| | | | ResolvinT4 |
| Docosahexaenoic | 17(S)-hydroxy- | Lipoxygenase-5 | ResolvinD1 |
| Acid | hydroperoxide | (LOX5) | (RvD1) |
| (DHA) | docosahexaenoic acid | Lipoxygenase-15 | ResolvinD2 |
| | (HpDHA) | (LOX15) | ResolvinD3 |
| | 4(S)-hydroperoxy-17(S) | Cycloxygenase-2 | ResolvinD4 |
| | hydroxy- | (COX2)/Aspirin | ResolvinD5 |
| | docosahexaenoic acid | | ResolvinD6 |
| | 4(S)-5(S)-epoxy-17(S)- | | Aspirin- |
| | hydroxy- | | triggered |
| | docosahexaenoic acid | | ResolvinD1 |
| | 17(S)-hydroxy- | | (At-RvD1) |
| | docosahexaenoic acid | | |
| | 7(S),8(S)-epoxy-17(S)- | | |
| | hydroxy- | | |
| | docosahexaenoic acid | | |
| | 17(S)-hydroxy- | Lipoxygenase-5 | Protectin |
| | hydroperoxide | (LOX5) | (PDX) |
| | docosahexaenoic acid | Lipoxygenase-15 | PCTR1 |
| | (HpDHA) | (LOX15) | PCTR2 |
| | 16(S),17(S)-epoxy | | PCTR3 |
| | Protectin | | |
| | 13(S),14(S)-epoxy- | Lipoxygenase-12 | Maresin1 |
| | Maresin | (LOX12) | (MaR1) |
| | 14(S)-hydroperoxy- | Lipoxygenase-15 | Maresin2 |
| | docosahexaenoic acid | (LOX15) | MCTR1 |
| | | Hydrolase | MCTR2 |
| | | Soluble Epoxide | MCTR3 |
| | | Hydrolase | |
| Eicosapentaenoic | 15(S)-hydroperoxy- | Lipoxygenase-5 | ResolvinE1 |
| Acid | eicosapentaenoic acid | (LOX5) | (RvE1) |
| (EPA) | 5(S)-Hydroperoxy- | Lipoxygenase-15 | ResolvinE2 |
| | 15(S)- | (LOX15) | ResolvinE3 |
| | hydroxyeicosapentanoic | Cycloxygenase-2 | ResolvinE4 |
| | acid | (COX2) | |
| | 18(R)-hydroperoxy- | P450 | |
| | eicosapentaenoic acid | | |
| | 5(S)-hydroperoxy- | | |
| | 18(R)- | | |
| | hydroxyeicosapentanoic | | |
| | acid | | |
| | 5(S),6(S)-epoxy-18(R)- | | |
| | hydroxyeicosapentanoic | | |
| | acid | | |

In some examples, the method may further include collecting conditioned media from the MSCs that have been contacted with the one or more SPM fatty acid precursors or SPM intermediates, or a combination of two or more thereof. In some examples, the MSCs are contacted with one or more SPM fatty acid precursors or SPM intermediates, or a combination of two or more thereof for about 3-48 hours (for example, about 3 hours, about 6 hours, about 12 hours, about 18 hours, about 24 hours, about 36 hours, or about 48 hours) prior to collecting the conditioned media. In one example, the MSCs are contacted with one or more SPM fatty acid precursors or SPM intermediates, or a combination of two or more thereof for about 24 hours prior to collecting the conditioned media.

### III. Pharmaceutical Compositions

Also provided are compositions that include MSCs that have been stimulated to increase production of SPMs, as described above. The composition includes stimulated MSCs and a carrier. The MSCs may be in suspension (e.g., in a solution). Appropriate solutions may include pharmaceutically and physiologically acceptable fluids such as water, physiological saline, balanced salt solutions, aqueous dextrose, glycerol, or the like as a vehicle.

In other examples, the carrier may be a material carrier, such as a biocompatible material carrier. Material carriers can include hydrogels, ceramics, polymers, and sponges, among others and can take various structures. In some examples, the carrier may include alginate, polyethylene glycol, hyaluronic acid, or collagen. For example, the MSCs may be encapsulated with a biocompatible polymer, such as polyethylene glycol, alginate, collagen, or gelatin. In some examples, and without being bound by theory, the material carrier may enhance the spatiotemporal presentation of SPMs upon delivery in vivo as a therapy.

A variety of biological or synthetic solid matrix materials (e.g., solid support matrices, biological adhesives or dressings, and biological/medical scaffolds) are suitable for use as a material carrier. The matrix material is generally physiologically acceptable and suitable for use in vivo. Non-limiting examples of such physiologically acceptable materials include, but are not limited to, solid matrix materials that are biodegradable, such crosslinked or non-crosslinked alginate, polyethylene glycol, hydrocolloid, foams, collagen gel, collagen sponge, polyglycolic acid (PGA) mesh, polyglactin (PGL) mesh, and bioadhesives (e.g., fibrin glue and fibrin gel). The polymer can be poly(D,L-lactic-co-glycolic acid) (PLGA) (see Lu et al., J. Biomater Sci Polym Ed. 9(11): 1187-205, 1998). In other embodiments, the matric includes poly (L-lactic acid) (PLLA) and poly(D,L-lactic-co-glycolic acid) (PLGA), such as with a co-polymer ratio of about 90:10, 75:25, 50:50, 25:75, 10:90 (PLLA:PLGA) (see Thomson et al., J. Biomed. Mater Res. A 95:1233-42, 2010).

Suitable material carriers also include porous meshes or sponges formed of synthetic or natural polymers. A non-limiting example is a polymeric hydrogel (including, but not limited to shear-thinning or self-healing hydrogels). Natural polymers that can be used include proteins such as collagen, albumin, and fibrin; and polysaccharides such as alginate and polymers of hyaluronic acid. Synthetic polymers can be biodegradable. Examples of biodegradable polymers include polymers of hydroxy acids such as polylactic acid (PLA). polyglycolic acid (PGA), and polylactic acid-glycolic acid (PLGA), polyorthoesters, polyanhydrides, polyphosphazenes, and combinations thereof.

In some implementations, the MSCs are encapsulated in a hydrogel. Cell encapsulation involves immobilization of cells in a polymeric semi-permeable membrane. A potential advantage of encapsulation of the MSCs is to improve or increase sustained or continuous release of SPMs at the site of administration over a period of time, such as at least 24 hours, at least 48 hours, at least 72 hours, at least 96 hours, at least 120 hours, or more (such as 1-3 days, 2-6 days, 5-10 days, 7 to 14 days, or longer). In some examples, the polymer is polyethylene glycol, alginate, cellulose sulfate, collagen, hyaluronic acid, fibrin, chitosan, gelatin, or agarose. In some examples, the polymer is polyethylene glycol. In other examples, the polymer is alginate, such as sodium alginate (see, e.g., Mckinney et al., Eur. Cell Mater. 37:42-59, 2020).

In some examples, the MSCs are encapsulated in a hydrogel including a functionalized polymer, such as functionalized PEG. In one example, the functionalized polymer is four-arm maleimide-functionalized PEG (PEG-4MAL). In some examples, the functionalized PEG is reacted with a thiol-containing “adhesive” peptide, such as an RGD peptide (for example, SEQ ID NO: 1). The RGD functionalized PEG macromers are subsequently cross-linked (for example, in the presence of MSCs, and optionally one or more SPM fatty acid precursors and/or one or more SPM intermediates) with a cross-linking agent. In one example, the crosslinking agent is a dithiol protease-cleavable VPM peptide (for example, SEQ ID NO: 2). In some examples, the RGD functionalized macromers as the VPM peptide are present at a 1:1 ratio. Exemplary methods for producing encapsulated MSCs are described in Example 2. See also, Phelps et al., Adv. Mater. 24:64-70, 2012. One of ordinary skill in the art can identify other methods and ratios for use in preparing encapsulated MSCs (with or without one or more SPM fatty acid precursors and/or one or more SPM intermediates),

In some examples, the encapsulated MSCs are treated with one or more SPM fatty acid precursors, SPM intermediates, or both. In some examples, following encapsulation of MSCs in a hydrogel, the hydrogel is treated with one or more SPM fatty acid precursors and/or one or more SPM intermediates, for example by incubating the encapsulated MSCs in a culture medium including the one or more SPM fatty acid precursors (such as DHA) and/or one or more SPM intermediates.

In additional examples, MSCs are co-encapsulated with one or more SPM fatty acid precursors, one or more SPM intermediates, or both. The MSCs may be unstimulated cells or may be MSCs that have been stimulated with one or more SPM fatty acid precursors, SPM intermediates, or both prior to encapsulation. In a particular example, MSCs are co-encapsulated with DHA. Without being bound by theory, it is believed that co-encapsulation of the MSCs (whether stimulated or unstimulated) with the one or more SPM fatty acid precursors and/or intermediates may increase production of SPMs (such as amount, duration, or both) by the MSCs compared to MSCs that are not co-encapsulated with the one or more SPM fatty acid precursors and/or intermediates.

### IV. Methods of Treatment

Provided herein are methods of treating disease or injury using the disclosed stimulated MSCs or conditioned media. In some examples, the methods include treating or inhibiting inflammation in a subject, including administering to the subject MSCs that have been stimulated to increase production of SPMs, as described above. In other examples, the methods include administering to the subject conditioned media from the stimulated MSCs.

In some examples where the subject is administered stimulated MSCs or a composition including stimulated MSCs, the MSCs are contacted with the one or more SPM fatty acid precursors or SPM intermediates, or a combination of two or more thereof for a sufficient period of time to stimulate or increase SPM production prior to administering to the subject. In some examples, the MSCs are contacted with one or more SPM fatty acid precursors or SPM intermediates, or a combination of two or more thereof for about 3-48 hours prior to administration (for example, about 3 hours, about 6 hours, about 12 hours, about 18 hours, about 24 hours, about 36 hours, or about 48 hours prior to administering to the subject).

In other examples, MSCs and the agent that stimulates SPM production (such as one or more SPM fatty acid precursors, SPM intermediates, or a combination of two or more thereof) are administered to the subject separately, thereby stimulating SPM production in vivo (e.g., rather than ex vivo). In some examples, the subject is administered unstimulated MSCs and the agent that stimulates SPM production simultaneously, substantially simultaneously, or sequentially. For example, the subject is administered unstimulated MSCs, followed by administration of the agent that stimulates SPM production, or vice versa. In some examples, the unstimulated MSCs and the agent that stimulates SPM production are administered at or near the same site (for example, both are administered intra-articularly to the same joint). In other examples, the unstimulated MSCs are administered at or near a site to be treated (e.g., intra-articularly) and the agent that stimulates SPM production is administered systemically.

In other examples, the subject is administered stimulated MSCs and an agent that stimulates SPM production separately. In some examples, the subject is administered stimulated MSCs and the agent that stimulates SPM production simultaneously, substantially simultaneously, or sequentially. For example, the subject is administered stimulated MSCs, followed by administration of the agent that stimulates SPM production, or vice versa. In some examples, the stimulated MSCs and the agent that stimulates SPM production are administered at or near the same site (for example, both are administered intra-articularly to the same joint). In other examples, the stimulated MSCs are administered at or near a site to be treated (e.g., intra-articularly) and the agent that stimulates SPM production is administered systemically.

In some examples, the subject has inflammation resulting from a musculoskeletal disorder or injury (for example, osteoarthritis, degenerative disc disease, joint injury, bone fracture, or muscle tear), an autoimmune disorder, or a dermal injury. Autoimmune diseases or disorders include diseases or conditions in which the immune system responds to self-antigens (autoreactive immune cells) resulting in self-destruction of healthy tissue. In some examples, the subject has an autoimmune disease or disorder selected from rheumatoid arthritis, graft-versus-host disease, Crohn's disease, arthritis, inflammatory bowel disease, or scleroderma.

In some examples, the MSCs are autologous to the subject being treated. Thus, in some examples, the methods further include collecting MSCs from the subject. In other examples, the MSCs are from a donor, such as an HLA-matched donor, or an unrelated mismatched donor. Thus in some examples, the methods further include collecting, isolating, and/or preparing MSCs from the subject being treated or from a donor.

The MSCs (or compositions including the MSCs, such as encapsulated cells) or conditioned media may be administered to the subject at or near the site of inflammation or injury. In some examples, the MSCs or conditioned media are injected in a joint (for example, intra-articularly), for example, in a subject with osteoarthritis or rheumatoid arthritis. In other examples, the MSCs or conditioned media are injected at or near a degenerating disc. In further examples, the MSCs are implanted at the site of an injury (such as a bone fracture or muscle, tendon, or ligament tear), for example with a carrier, such as a sponge. In additional examples, the MSCs or conditioned media may be applied on a patch or bandage, for example, in the case of a dermal injury. In other examples, the MSCs or conditioned media may be administered to the subject systemically (for example, intravenously).

In some examples, the subject is administered one dose of the MSCs or conditioned media, while in other examples, the subject is administered two or more (such as 2, 3, 4, 5, or more) doses. For example, the subject may be administered the MSCs or conditioned media weekly, every other week, every 3 weeks, monthly, every 2 months, every 3 months, every 6 months, or yearly. A skilled clinician can select appropriate dose(s) and route(s) of administration based on factors such as the disease or disorder being treated, the condition of the subject, and other considerations.

### EXAMPLES

The following examples are provided to illustrate particular features of certain aspects of the disclosure, but the scope of the disclosure should not be limited to those features exemplified.

### Example 1

### Enhancing Production of Specialized Pro-Resolving Mediators by Mesenchymal Stromal Cells

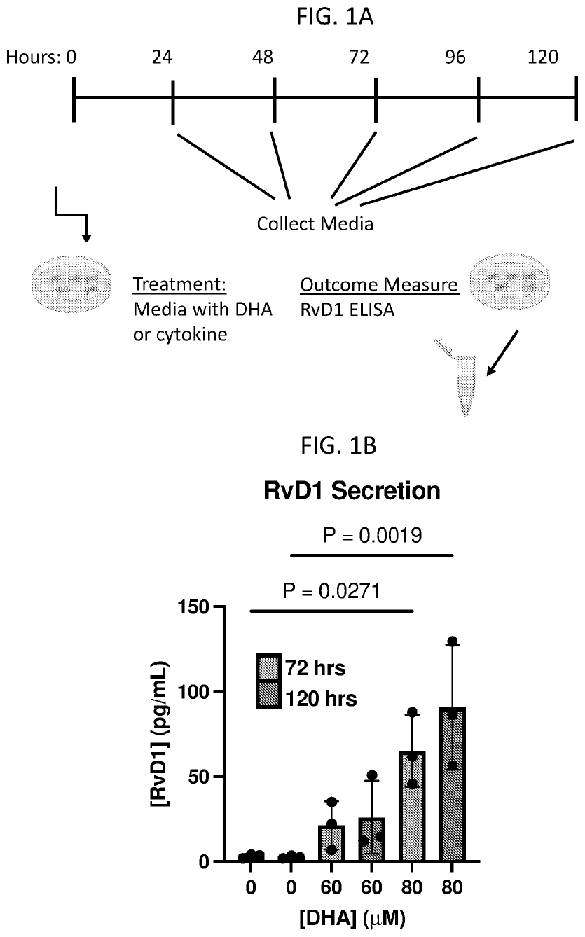

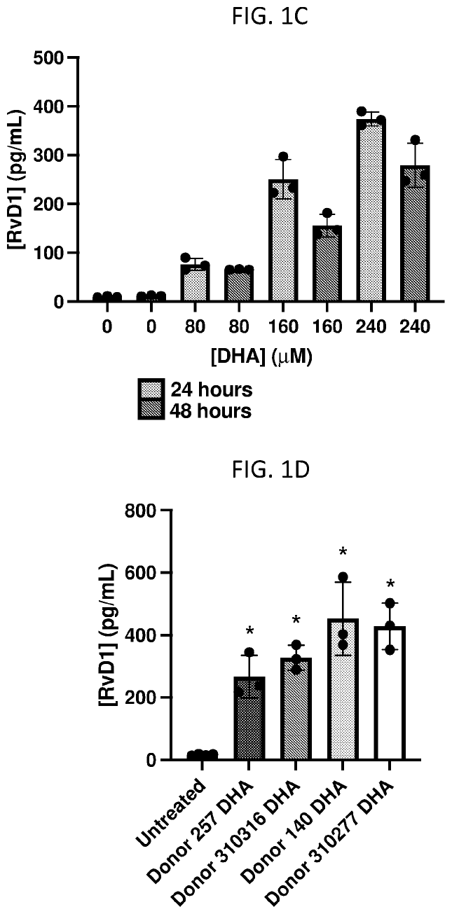

Human bone marrow-derived Mesenchymal Stromal Cells (MSCs) were obtained from RoosterBio (Frederick, MD) and cultured in RoosterBasal Media in 5% carbon dioxide at 37° C. After MSC culture reached ˜70% confluency, DHA supplemented media was added at either 60, 80, 160, or 240 μM DHA concentration (FIG. 1A). Samples were collected at different time points and analyzed with RvD1 ELISA assay kit (Cayman Chemical, Ann Arbor, MI). hMSCs treated with DHA biosynthesized and secreted RvD1 in a dose-dependent manner, with higher concentrations at shorter time points (FIGS. 1B and 1C).

Previous studies have shown high donor variability in the secretory profile and therapeutic efficacy of MSCs. To determine whether these inconsistences affect SPM production in MSCs, RvD1 biosynthesis was measured after 24 hours of DHA treatment (80 μM) in MSCs from four different donors. All cells produced RvD1 at levels significantly higher than untreated control hMSCs (from donor 257) (FIG. 1D). These results demonstrate reliable MSC synthesis of RvD1 upon precursor treatment from multiple different donors.

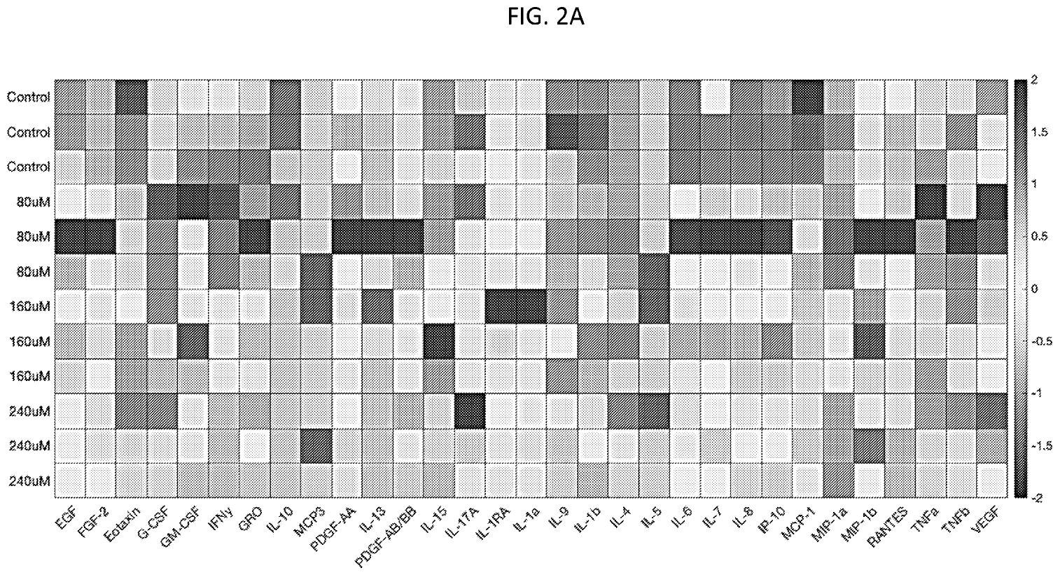

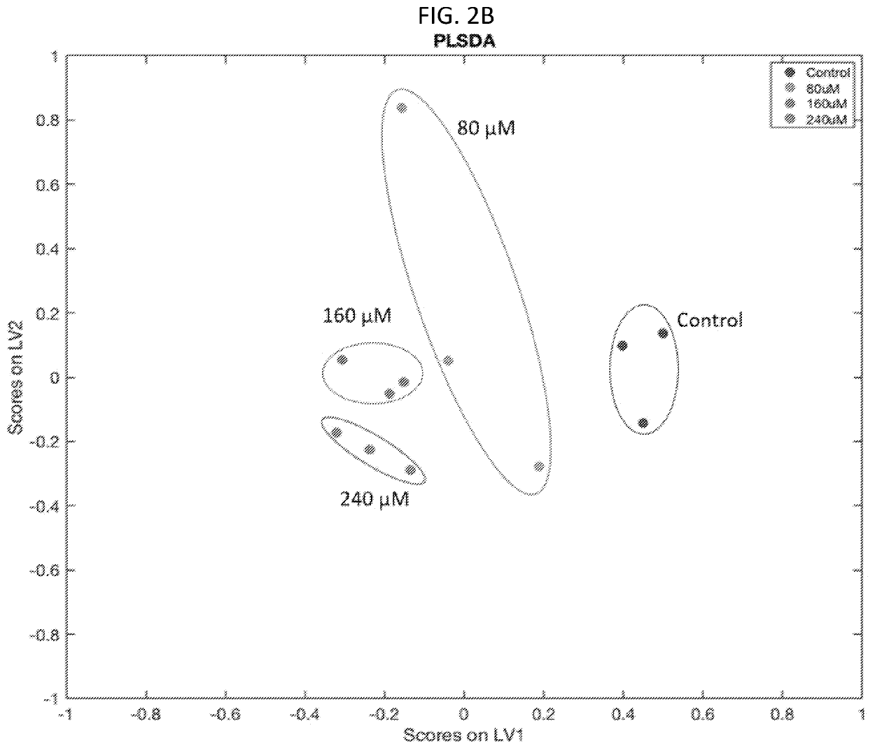

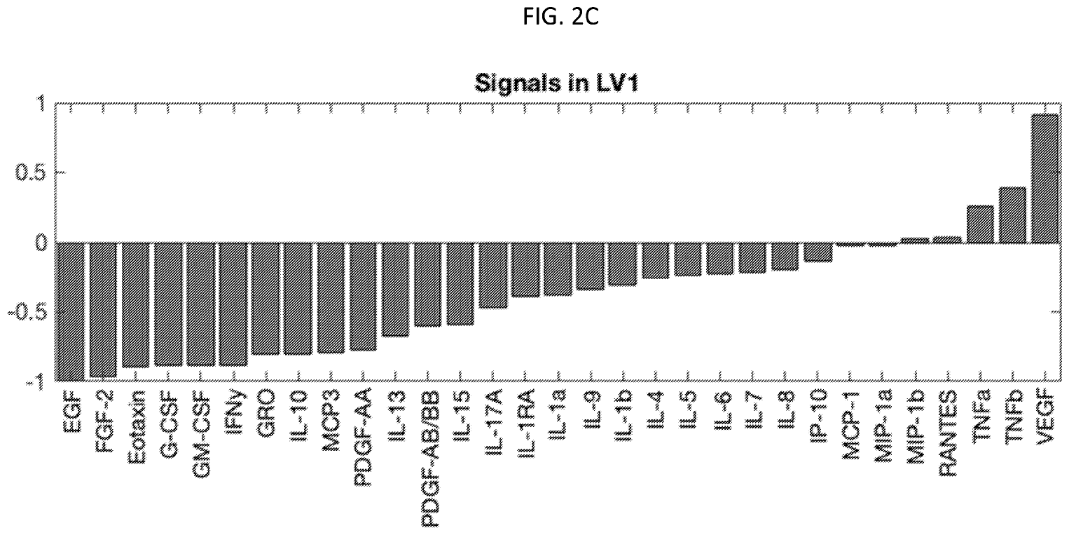

For cytokine secretome analysis, MSCs were cultured as stated above and samples were collected after 24 hrs of DHA treatment. Cytokine levels were analyzed with Milliplex Human Cytokine/Chemokine Magnetic Bead Multiplex kit (Millipore, Burlington, MA). There was distinct separation of the hMSC groups according to LV1 and cytokine levels broadly increased in DHA-treated hMSCs, including GM-CSF, GRO, and IL-4 among others (FIGS. 2A-2C).

### Example 2

### Production of SPMs by Hydrogel Encapsulated MSCs

MSCs were cultured in Tissue Culture Plastic (TCP) or encapsulated in PEG-4MAL hydrogels. The 20 kDa four-arm PEG-4MAL was sourced from Laysan Bio (Item #: 4arm-PEG-20K-5g). A solution of 4% w/v PEG in a 0.5 M (pH=7) MES buffer was prepared. The two peptides, RGD (GCGYGRGDSPG; SEQ ID NO: 1) for functionalization of the gel and VPM (GCRDVPMSMRGGDRCG; SEQ ID NO: 2) for crosslinking were also dissolved in the 0.5 M MES buffer. The hydrogels were prepared using a 2:1:1:1 volume ratio of PEG: RGD: cells/PBS: VPM. The 4% PEG translates to 2 mM in the hydrogel prepared from a 5 mM PEG stock solution. Based on the concentration of maleimides per PEG molecule (8 mM maleimide in 2 mM PEG-4MAL) the RGD concentration was 0.768 mM final concentration in the gel (from a 3.84 mM stock and accounting for ˜96% purity) and the VPM concentration required to cross-link the remaining maleimides in the PEG was 3.456 mM final in the gel (17.28 mM stock).

Silicon molds were used to prepare 50 μL of 2 mM gels by mixing the PEG, RGD, and cells or PBS initially, then adding the VPM at the last step. The MSC density in these cells was 1×10⁶ cells/mL in 50 μL hydrogels. Addition of VPM initiates rapid cross linking and the solution forms a soft gel within a minute. Ten minutes after cross-linking, the gels were removed from their molds and placed in 24-well cell culture dishes that are designed to repel cell attachment (Ultra-Low Attachment plates by Corning) and cell culture medium was added.

MSCs were treated with 160 μM DHA in the hydrogels in three different conditions. DHA was added to the cell culture media immediately after gel formation in Group A. In Group B, DHA was added to the cell culture media after the cells were allowed to proliferate in the gel for 24 hours. Samples of cell culture media were taken incrementally over a period of five days and Resolvin D1 levels were measured via ELISA assay. In addition, samples of the cell culture medium were taken 24 hours after treatment for each group and these samples were analyzed by LCMS (Wayne State University Lipidomics core facility).

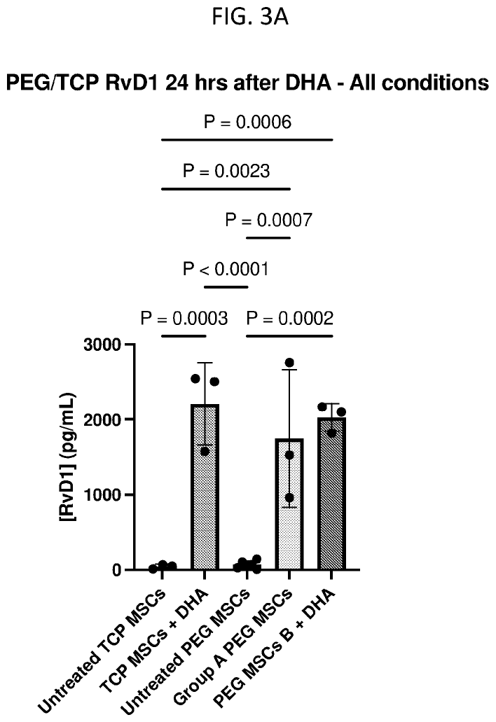

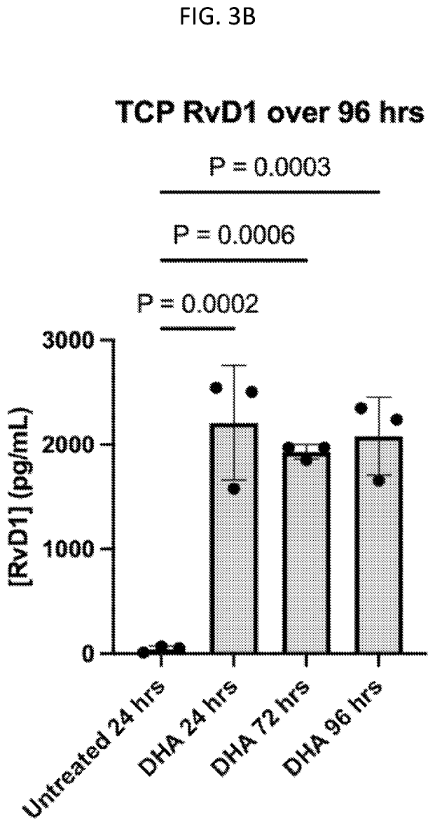

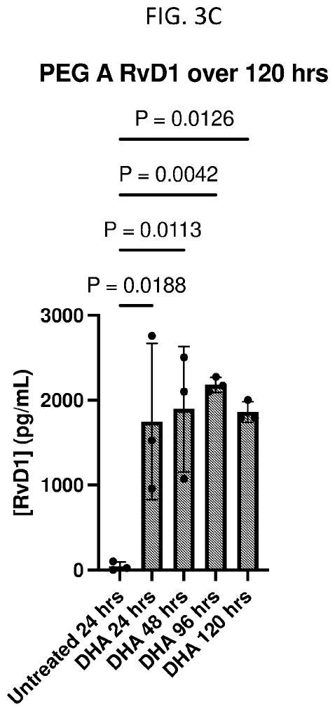

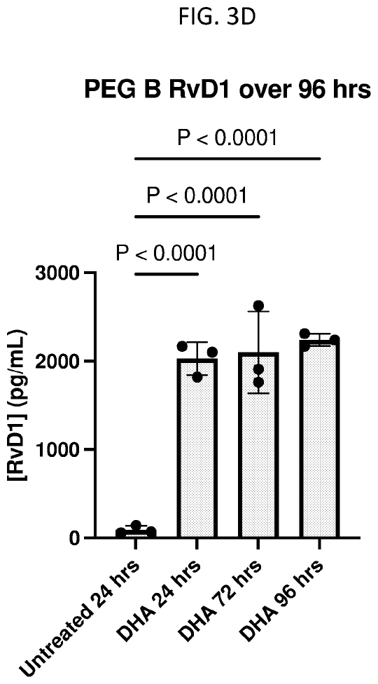

These data show that MSCs encapsulated in PEG-4MAL hydrogels and treated with DHA had significantly increased RvD1 production compared with untreated cells (FIG. 3A). The MSCs continued to produce RvD1 for at least 120 hours (FIGS. 3B-3D), suggesting that PEG-4MAL encapsulation could result in a sustained-release RvD1 environment for the MSCs.

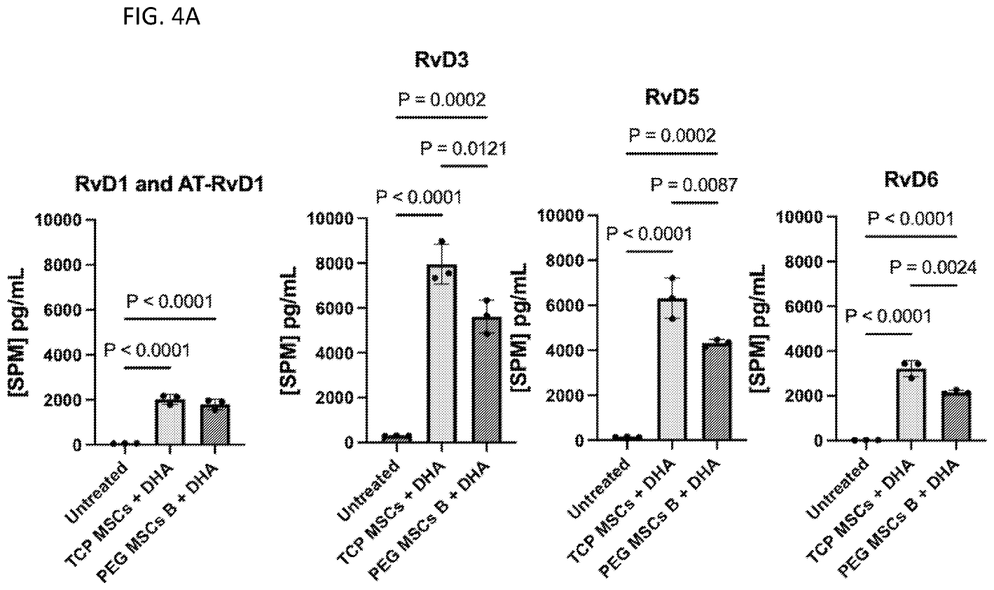

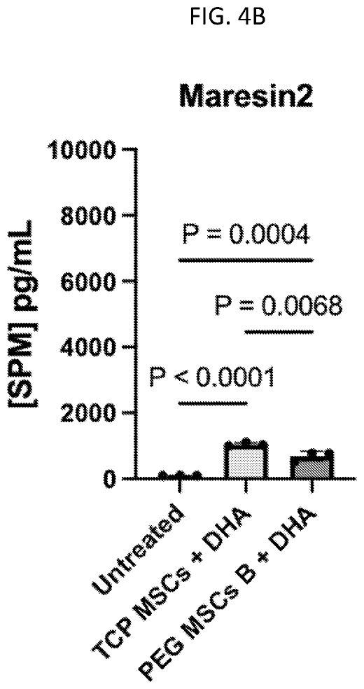

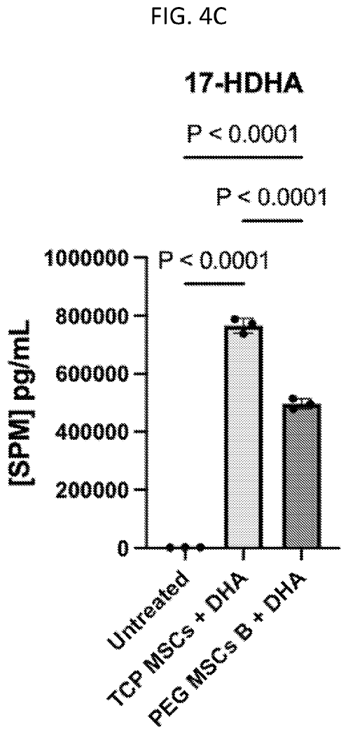

Lipidomics LCMS analysis allows for quantitative measurement of lipid concentrations and allows for analysis of a broad panel of lipids, instead of a single analyte as in an ELISA assay. These data confirmed that DHA-treated MSCs have a significant increase in D-series Resolvin production including Resolvin D1, D3, D5 and D6 (FIG. 4A). Resolvins were produced and secreted by MSCs in both the 2D and the 3D culture environments. These data also show that the 17-HDHA intermediate in the enzymatic pathway for D-series Resolvin synthesis from DHA was present in media from treated cells at abundant levels (FIG. 4C). The DHA treated MSCs also showed a significant increase in the production of Maresin2 (FIG. 4B).

### Example 3

### Methods of Testing Efficacy of Resolvin-Secreting MSCs in a Model of Osteoarthritis

This example describes particular methods that can be used to evaluate the efficacy of resolvin-secreting MSCs for treating osteoarthritis. One skilled in the art will appreciate that methods that deviate from these specific methods can also be used to successfully evaluate the efficacy of such MSCs.

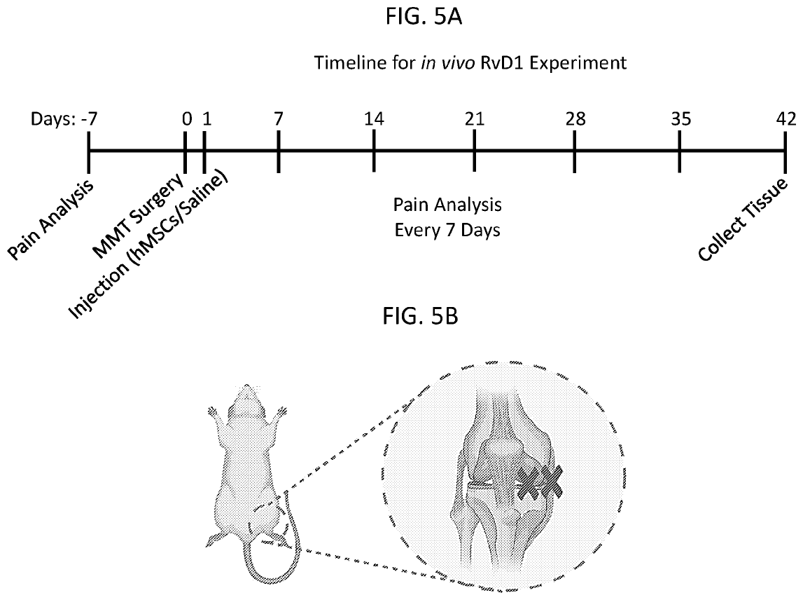

A schematic diagram of the method is provided in FIG. 5A. Medial meniscal transection (MMT) (FIG. 5B), which leads to OA symptoms, is utilized as the model.

Medial meniscal transection surgery: Briefly, 12-week old male Lewis rats will be anesthetized with isoflurane. An incision will be made in the skin of the left knee and the medial collateral ligament (MCL) will be exposed and transected. For MMT, the meniscus will be cut through the full thickness at its narrowest point. The joint will be irrigated with normal saline, the capsule sutured with 4-0 vicryl, and the skin closed with wound clips. Sham animals will undergo identical surgical manipulation as an MMT animal except the meniscus is not transected.

hMSC culture, DHA treatment, and intra-articular injection: MSCs will be cultured in vitro in RoosterNourish-MSC-XF (Cat #: XF KT-016, RoosterBio, Frederick, MD, USA) and used up to a population doubling limit of 20. DHA treatment will be carried out by switching cells to media with 160 μM DHA added once cells are ˜70% confluent. Samples of media will be collected 24 hours after DHA treatment and varying later time points. Resolvin D1 levels will be analyzed with an ELISA kit that specifically recognizes Resolvin D1 (Item #500380, Cayman Chemical, 1180 East Ellsworth Rd., Ann Arbor, MI, USA). Quantification via ELISA will be verified using lipidomic LCMS. MSCs will be prepared for intra-articular injection by culturing in T225 flasks and treating them with 160 μM DHA 24 hours prior to injection. Immediately before injections are carried out, MSCs will be lifted with TrypLE Select Enzyme (Gibco, catalog #12563011), pelleted at 200 rcf for 10 minutes, then resuspended to a concentration of 10,000 cells/μL in Hank's Balanced Salt Solution (ThermoFisher catalog #J67771-AP). Each injection will be a volume of 50 μL. containing 500,000 cells and administered intra-articularly to anesthetized animals using a 25-Gauge needle.

Tactile Allodynia Measurements Using Von Frey Filaments: Tactile allodynia measurements of pain sensitivity the hindlimbs will be made using Von Frey filaments according to published protocols. Briefly Von Frey filaments (Stoelting, Wood Dale, IL, USA) will be applied through a wire cage to the plantar surface of the rats' hind paws. The smallest filament to induce withdrawal will be recorded for the paw withdrawal threshold measurement, which is indicative of pain sensitivity.

Dynamic Weight Bearing testing: Pain sensitivity and limb function will be assessed using the Dynamic Weight Bearing (DWB) Testing System. The DWB test allows paw identification for dynamic weight distribution on all paws for assessment of pain sensitivity in in vivo models. Measurements will be taken at baseline (prior to MMT surgery), and weekly post-injury. The DWB device consists of a plexiglass chamber with a sensor pad on the floor and a high-resolution video camera centrally located in the lid of the chamber. During testing the animal is placed on the sensor pad in the chamber and allowed to freely move about the chamber without restraint for at least 5 minutes. The force exerted by each paw on the sensor pad and video data are recorded in real-time and autoscored by the DWB software at the end of the testing period. Data parameters collected during the testing period includes force applied by each of the paws on the sensor pad, surface area of each paw, and total time spent on each paw in a specific posture. The autoscored data is analyzed, results data generated and exported using the analysis function within the DWB software. Using this software, pain sensitivity outcomes will be evaluated as weight bearing difference between the hindlimbs while both limbs are in contact with the sensor pad.

EPIC-μCT analysis: EPIC-μCT is a non-destructive technique, and the same samples will also be used for histology. The hindlimbs will be harvested and fixed in 10% neutral buffered formalin at designated timepoints. Tibiae will be dissected free and prepared for EPIC-μCT analysis. Briefly, tibiae will be immersed in 2 ml of 30% Hexabrix™ 320 contrast agent (Covidien, Hazelwood, MO) and 70% PBS at 37° C. for 30 minutes. Samples will then be scanned using a μCT 40 (Scanco Medical, Bruttisellen, Switzerland) at 45kVp, 177 μA, 200 ms integration time, and a voxel size of 16 μm. Coronal sections of the cartilage will be contoured and thresholded with global segmentation parameters. Outcome measures will include average articular cartilage thickness, volume and attenuation. Cartilage attenuation is a quantitative parameter that is inversely proportional to sulfated glycosaminoglycan (sGAG) content (degraded cartilage will have higher contrast agent and thus higher attenuation values). Focal lesion defects will be evaluated for incidence of lesion and lesion volume. Osteophytes will be analyzed for cartilage volume, mineralized volume, and tissue thickness. Lastly, subchondral bone will be contoured and thresholded from the articular cartilage and underlying trabecular bone. Subchondral bone thickness, volume, and mineral density will be measured.

Histopathology of Synovium, Cartilage and Bone: After EPIC-pCT, tibiae and femora will be decalcified in ImmunoCal (Decal Chem Co, Tallman, NY) for 7 days. Samples will be embedded into paraffin blocks and serial sections will be cut at 5 μm thickness. Serial sections will be stained for sGAGs using 0.5% Safranin-O (Saf-O) solution and counterstained with 0.2% aqueous FastGreen solution; alternatively, sections will be stained with hematoxylin and eosin. Sections will be scored according to OARSI guidelines established by Gerwin et al. (Osteoarthritis Cartilage 18(Suppl 3):S24-34, 2010) and Willett et al. (Osteoarthritis Cartilage 24:1604-1612, 2016). Sections will be blinded, analyzed microscopically and scored by trained reviewers. For each knee, three sections will be scored and averaged. Scores will include all parameters detailed by OARSI guidelines, including (but not limited to) cartilage degeneration, synovitis, subchondral bone sclerosis and osteophyte analysis.

Statistics: Statistical comparisons will be made for all parameters described above between Sham-, MMT- and contralateral hindlimbs using analysis of variance (ANOVA, p<0.05) and Mann-Whitney U-tests.

Outcomes: It is expected that animals that receive saline injections will continue to develop pain and favor their uninjured limbs, while those injected with the DHA-treated MSCs will approach pain and weight-bearing levels similar to the sham animals. Those injected with untreated MSCs are expected to also approach sham animal levels, but to a lesser extent. Furthermore, it is expected that the animals that receive therapeutic injections will have smoother tibial plateaus, fewer osteophytes and less cartilage thickening and attenuation.

It will be apparent that the precise details of the methods or compositions described may be varied or modified without departing from the spirit of the described aspects of the disclosure. We claim all such modifications and variations that fall within the scope and spirit of the claims below.

1. A method of stimulating or increasing production of one or more specialized pro-resolving mediators (SPMs) by mesenchymal stromal cells (MSCs), comprising contacting a population of MSCs with one or more SPM fatty acid precursors or SPM intermediates, or a combination of two or more thereof,
thereby stimulating or increasing production of SPMs.

2. The method of claim 1, wherein:
the one or more SPM fatty acid precursors is selected from the group consisting of docosahexaenoic acid (DHA), eicosapentaenoic acid (EPA), arachidonic acid (AA), and docosapentaenoic acid (DPA); or
the one or more SPM intermediates is selected from the group consisting of 17(S)-hyrdoxy-docosahexaenoic acid, 17(S)-hydroxy-hydroperoxide docosahexaenoic acid (HpDHA), 4(S)-hydroperoxy-17(S) hydroxy-docosahexaenoic acid, 4(S)-5(S)-epoxy-17(S)-hydroxy-docosahexaenoic acid, 7(S),8(S)-epoxy-17(S)-hydroxy-docosahexaenoic acid, 15(S)-hydroxy-docosahexaenoic acid, 5(S)-Hydroperoxy-15(S)-hydroxyeicosapentanoic acid, 18(R)-hydroperoxy-eicosapentaenoic acid, 5(S)-hydroperoxy-18(R)-hydroxyeicosapentanoic acid, 5(S),6(S)-epoxy-18(R)-hydroxyeicosapentanoic acid, 15(S)-Hydroperoxy eicosapentanoic acid, 5(S),15(S)-Di-Hydroperoxide eicosatetraenoic acid, 5(S),6(S)-epoxy-15(S) Di-Hydroperoxide eicosatetraenoic acid, and 13(R)-Hydroperoxy docosapentaenoic acid (HpDPA).

3-4. (canceled)

5. The method of claim 1, wherein the SPMs are selected from one or more resolvins, lipoxins, protectins, maresins, or a combination of two or more thereof.

6. The method of claim 5, wherein:
the one or more resolvins are selected from the group consisting of resolvin D1, resolvin D2, resolvin D3, resolvin D4, resolvin D5, resolvin D6, aspirin-triggered resolvin D1, resolvin E1, resolvin E2, resolvin E3, resolvin E4, resolvin T1, resolvin T2, resolvin T3, and resolvin T4;
the one or more lipoxins are selected from the group consisting of lipoxin A₄, lipoxin A₅, lipoxin B₄, and 15-epi-lipoxin A₄;
the one or more protectins are selected from the group consisting of protectin (PDX), PCTR1, PCTR2, and PCTR3; or
the one or more maresins are selected from the group consisting of maresin 1, maresin 2, MCTR1, MCTR2, and MCTR3.

7-12. (canceled)

13. The method of claim 1, further comprising collecting conditioned media from the MSCs contacted with the one or more SPM fatty acid precursors, SPM intermediates, or a combination of two or more thereof.

14. A composition comprising MSCs produced by the method of claim 1 and a pharmaceutically acceptable carrier.

15. The composition of claim 14, wherein the carrier comprises physiological saline, a balanced salt solution, a hydrogel, ceramic, polymer, sponge, or scaffold.

16-18. (canceled)

19. A method of treating or inhibiting inflammation in a subject, comprising administering to the subject:
the composition of claim 14.
thereby treating or inhibiting the inflammation in the subject.

20. A method of treating or inhibiting inflammation in a subject, comprising administering to the subject:
mesenchymal stromal cells (MSCs) and one or more specialized pro-resolving molecule (SPM) fatty acid precursors or SPM intermediates, or a combination of two or more thereof,
thereby treating or inhibiting the inflammation in the subject.

21. The method of claim 20, wherein the MSCs and the one or more specialized pro-resolving molecule (SPM) fatty acid precursors or SPM intermediates, or a combination of two or more thereof are administered to the subject simultaneously, substantially simultaneously, or sequentially.

22. The method of claim 20, wherein;
the one or more SPM fatty acid precursors is selected from the group consisting of docosahexaenoic acid (DHA), eicosapentaenoic acid (EPA), arachidonic acid (AA), and docosapentaenoic acid (DPA); or
the one or more SPM intermediates is selected from the group consisting of 17(S)-hyrdoxy-docosahexaenoic acid, 17(S)-hydroxy-hydroperoxide docosahexaenoic acid (HpDHA), 4(S)-hydroperoxy-17(S) hydroxy-docosahexaenoic acid, 4(S)-5(S)-epoxy-17(S)-hydroxy-docosahexaenoic acid, 7(S),8(S)-epoxy-17(S)-hydroxy-docosahexaenoic acid, 15(S)-hydroxy-docosahexaenoic acid, 5(S)-Hydroperoxy-15(S)-hydroxyeicosapentanoic acid, 18(R)-hydroperoxy-eicosapentaenoic acid, 5(S)-hydroperoxy-18(R)-hydroxyeicosapentanoic acid, 5(S),6(S)-epoxy-18(R)-hydroxyeicosapentanoic acid, 15(S)-Hydroperoxy eicosapentanoic acid, 5(S),15(S)-Di-Hydroperoxide eicosatetraenoic acid, 5(S),6(S)-epoxy-15(S) Di-Hydroperoxide eicosatetraenoic acid, and 13(R)-Hydroperoxy docosapentaenoic acid (HpDPA).
23-24. (canceled)

25. The method of claim 20, wherein the SPMs are selected from one or more resolvins, lipoxins, protectins, maresins, or a combination of two or more thereof.

26. The method of claim 25, wherein:
the one or more resolvins are selected from the group consisting of resolvin D1, resolvin D2, resolvin D3, resolvin D4, resolvin D5, resolvin D6, aspirin-triggered resolvin D1, resolvin E1, resolvin E2, resolvin E3, resolvin E4, resolvin T1, resolvin T2, resolvin T3, and resolvin T4;
the one or more lipoxins are selected from the group consisting of lipoxin A₄, lipoxin A₅, lipoxin B₄, and 15-epi-lipoxin A₄;
the one or more protectins are selected from the group consisting of protectin (PDX), PCTR1, PCTR2, and PCTR3; or
the one or more maresins are selected from the group consisting of maresin 1, maresin 2, MCTR1, MCTR2, and MCTR3.

27-30. (canceled)

31. The method of claim 20, wherein the MSCs, the one or more specialized pro-resolving molecule (SPM) fatty acid precursors or SPM intermediates, or a combination of two or more thereof, or both are formulated in a pharmaceutically acceptable carrier.

32. The method of claim 31, wherein the carrier comprises physiological saline, a balanced salt solution, a hydrogel, ceramic, polymer, sponge, or scaffold.

33. (canceled)

34. The method of claim 32, wherein the carrier is a hydrogel comprising polyethylene glycol or polyethylene glycol functionalized with one to four maleimide moieties.

35-36. (canceled)

37. A method of treating or inhibiting inflammation in a subject, comprising administering to the subject conditioned media from MSCs produced by the method of claim 13, thereby treating or inhibiting the inflammation in the subject.

38. The method of claim 12, wherein the subject has inflammation from a musculoskeletal disorder or injury, or a dermal injury.

39. (canceled)

40. The method of claim 19, wherein the MSCs are administered to the subject at or near the site of inflammation or injury.

41. (canceled)

42. The method of claim 19, wherein the MSCs are autologous to the subject.


SEQUENCE LISTING (2 sequences)

SEQ ID NO: 1 - protein, 11 aa, synthetic construct
GCGYGRGDSPG

SEQ ID NO: 2 - protein, 16 aa, synthetic construct
GCRDVPMSMRGGDRCG